(19) United States
(12) Patent Application Publication
(10) Pub. No.: US 2013/0022494 A1
(43) Pub. Date: Jan. 24, 2013

(54) METHOD AND SYSTEM FOR STERILIZING OBJECTS BY THE APPLICATION OF BEAM TECHNOLOGY

(71) Applicants: Sean R. Kirkpatrick, Littleton, MA (US); Richard C. Svrluga, Cambridge, MA (US); Joseph Khoury, Dedham, MA (US)

(72) Inventors: Sean R. Kirkpatrick, Littleton, MA (US); Richard C. Svrluga, Cambridge, MA (US); Joseph Khoury, Dedham, MA (US)

(73) Assignee: EXOGENESIS CORPORATION, Billerica, MA (US)

(21) Appl. No.: 13/358,213

(22) Filed: Jan. 25, 2012

Related U.S. Application Data
(63) Continuation-in-part of application No. 12/492,661, filed on Jun. 26, 2009.
(60) Provisional application No. 61/075,957, filed on Jun. 26, 2008.
(60) Provisional application No. 61/436,145, filed on Jan. 25, 2011.
(60) Provisional application No. 61/525,234, filed on Aug. 19, 2011.

(51) Int. Cl. A61L 2/08

(52) U.S. Cl. 422/22

(57) ABSTRACT

Methods and systems for sterilization of objects by gas-cluster ion-beam (GCIB) irradiation or by accelerated Neutral Beam are disclosed. The sterilization may be in conjunction with other beneficial GCIB surface processing of the objects. The objects may be medical devices or surgically implantable medical prostheses. The accelerated Neutral Beam is derived from an accelerated GCIB.

### CROSS REFERENCE TO RELATED APPLICATIONS

This application is a Continuation-in-Part application to U.S. patent application Ser. No. 12/492,661, filed Jun. 26, 2009, entitled METHOD AND SYSTEM FOR STERILIZING OBJECTS BY THE APPLICATION OF GAS CLUSTER ION BEAM TECHNOLOGY, which claims priority from U.S. Provisional Patent application Ser. No. 61/075,957, filed Jun. 26, 2008.

This application also claims priority from U.S. Provisional Patent Application Ser. No. 61/436,145, filed Jan. 25, 2011, titled METHOD AND SYSTEM FOR STERILIZING BY THE APPLICATION OF GAS-CLUSTER ION-BEAM TECHNOLOGY AND BIOLOGICAL MATERIALS STERILIZED THEREBY, and U.S. Provisional Patent Application Ser. No. 61/525,234, filed Aug. 19, 2011, titled METHOD AND SYSTEM FOR STERLIZING OBJECTS BY THE APPLICATION OF BEAM TECHNOLOGY and incorporated herein by reference in their entirety.

### FIELD OF THE INVENTION

This invention relates generally to the sterilization of objects including medical devices by irradiation with gas-cluster ion-beam (GCIB) or by irradiation with an accelerated Neutral Beam derived from an accelerated GCIB. The sterilization may be performed in combination with other GCIB or Neutral Beam processing of the object.

### BACKGROUND OF THE INVENTION

Sterilization of objects such as medical devices or surgically implantable devices or prostheses has traditionally been done by a variety of methods including steam or dry heating, ultraviolet, x-ray, or gamma-ray irradiation, plasma sterilization, conventional ion beam irradiation, and exposure to sterilant gases or germicidal fluids.

Gas-cluster ions are formed from large numbers of weakly bound atoms or molecules sharing common electrical charges and they can be accelerated to have high total energies. Gas-cluster ions disintegrate upon impact and the total energy of the cluster ion is shared among the constituent atoms. Because of this energy sharing, the atoms are individually much less energetic than in the case of un-clustered conventional ions and, as a result, the atoms only penetrate to much shallower depths than would conventional ions. Surface effects can be orders of magnitude stronger than corresponding effects produced by conventional ions, thereby making important micro-scale surface modification effects possible that are not possible in any other way.

The concept of gas-cluster ion-beam (GCIB) processing has only emerged in recent decades. Using a GCIB for dry etching, cleaning, and smoothing of materials, as well as for film formation is known in the art and has been described, for example, by Deguchi, et al. in U.S. Pat. No. 5,814,194, “Substrate Surface Treatment Method”, 1998. Because ionized gas-clusters containing on the order of thousands of gas atoms or molecules may be formed and accelerated to modest energies on the order of a few thousands of electron volts, individual atoms or molecules in the clusters may each only have an average energy on the order of a few electron volts. It is known from the teachings of Yamada in, for example, U.S. Pat. No. 5,459,326, that such individual atoms are not energetic enough to significantly penetrate a surface to cause the residual sub-surface damage typically associated with plasma polishing or conventional monomer ion beam processing. Nevertheless, the clusters themselves are sufficiently energetic (some thousands of electron volts) to effectively etch, smooth, or clean hard surfaces, or to perform other shallow surface modifications.

Because the energies of individual atoms within a gas-cluster ion are very small, typically a few eV, the atoms penetrate through only a few atomic layers, at most, of a target surface during impact. This shallow penetration of the impacting atoms means all of the energy carried by an entire cluster ion is consequently dissipated in an extremely small volume in the top surface layer during an extremely short time interval. This is different from the case of ion implantation, which is normally done with conventional ions and where the intent is to penetrate into the material, sometimes penetrating several thousand angstroms, to produce changes in both the surface and sub-surface properties of the material. Because of the high total energy of the cluster ion and extremely small interaction volume of each cluster, the deposited energy density at the impact site is far greater than in the case of bombardment by conventional ions and the extreme conditions permit material modifications not otherwise achievable.

Irradiation by GCIB has been successfully applied in a variety of surface modification processes including cleaning, smoothing, surface infusion, deposition, etching, and changing surface characteristics such as making a surface more or less wettable. The cleaning, smoothing, etching, and wettability modification processes (for example) are sometimes useful for improving the surfaces of medical devices, surgical implants, and medical prostheses. It is desirable and necessary that many types of medical devices, implants, and prostheses be sterile for use in their intended applications.

Ions have long been favored for many processes because their electric charge facilitates their manipulation by electrostatic and magnetic fields. This introduces great flexibility in processing. However, in some applications, the charge that is inherent to any ion (including gas cluster ions in a GCIB) may produce undesirable effects in the processed surfaces. GCIB has a distinct advantage over conventional ion beams in that a gas cluster ion with a single or small multiple charge enables the transport and control of a much larger mass-flow (a cluster may consist of hundreds or thousands of molecules) compared to a conventional ion (a single atom, molecule, or molecular fragment.) Particularly in the case of insulating materials, surfaces processed using ions often suffer from charge-induced damage resulting from abrupt discharge of accumulated charges, or production of damaging electrical field-induced stress in the material (again resulting from accumulated charges.) In many such cases, GCIBs have an advantage due to their relatively low charge per mass, but in some instances may not eliminate the target-charging problem. Furthermore, moderate to high current intensity ion beams may suffer from a significant space charge-induced defocusing of the beam that tends to inhibit transporting a well-focused beam over long distances. Again, due to their lower charge per mass relative to conventional ion beams, GCIBs have an advantage, but they do not fully eliminate the space charge transport problem.

A further instance of need or opportunity arises from the fact that although the use of beams of neutral molecules or atoms provides benefit in some surface processing applications and in space charge-free beam transport, it has not generally been easy and economical to produce intense beams of neutral molecules or atoms except for the case of nozzle jets, where the energies are generally on the order of a few milli-electron-volts per atom or molecule, and thus have limited processing capabilities. More energetic neutral particles can be beneficial or necessary in many applications, for example when it is desirable to break surface or shallow subsurface bonds to facilitate cleaning, etching, smoothing, deposition, amorphization, or to produce surface chemistry effects. In such cases, energies of from about an eV up to a few thousands of eV per particle can often be useful. Methods and apparatus for forming such Neutral Beams by first forming an accelerated charged GCIB and then neutralizing or arranging for neutralization of at least a fraction of the beam and separating the charged and uncharged fractions are disclosed herein. The Neutral Beams may consist of neutral gas clusters, neutral monomers, or a combination of both. Although GCIB processing has been employed successfully for many applications, there are new and existing application needs not fully met by GCIB or other state of the art methods and apparatus, and wherein accelerated Neutral Beams may provide superior results. For example, in many situations, while a GCIB can produce dramatic atomic-scale smoothing of an initially somewhat rough surface, the ultimate smoothing that can be achieved is often less than the required smoothness, and in other situations GCIB processing can result in roughening moderately smooth surfaces rather than smoothing them further.

It is therefore an object of this invention to provide methods and apparatus for surface sterilization of objects including medical devices, surgical implants, and/or medical prostheses by GCIB or Neutral Beam irradiation.

It is another object of this invention to provide methods and apparatus for multi-step processing of objects including a step of surface sterilization by GCIB or Neutral Beam irradiation in combination with another GCIB or Neutral Beam surface processing step on the same object.

It is a further object of this invention to provide methods and apparatus for surface sterilization of objects, without significantly elevating the temperature of the bulk of the object and without the use of toxic materials.

### SUMMARY OF THE INVENTION

The objects set forth above, as well as further and other objects and advantages of the present invention, are achieved as described hereinbelow.

Beams of energetic conventional ions, accelerated electrically charged atoms or molecules, are widely utilized to form semiconductor device junctions, to modify surfaces by sputtering, and to modify the properties of thin films. Unlike conventional ions, gas cluster ions are formed from clusters of large numbers (having a typical distribution of several hundreds to several thousands with a mean value of a few thousand) of weakly bound atoms or molecules of materials that are gaseous under conditions of standard temperature and pressure (commonly oxygen, nitrogen, or an inert gas such as argon, for example, but any condensable gas can be used to generate gas cluster ions) with each cluster sharing one or more electrical charges, and which are accelerated together through large electric potential differences (on the order of from about 3 kV to about 70 kV or more) to have high total energies. After gas cluster ions have been formed and accelerated, their charge states may be altered or become altered (even neutralized), and they may fragment or may be induced to fragment into smaller cluster ions or into monomer ions and/or neutralized smaller clusters and neutralized monomers, but they tend to retain the relatively high velocities and energies that result from having been accelerated through large electric potential differences, with the energy being distributed over the fragments. After gas cluster ions have been formed and accelerated, their charge states may be altered or become altered (even neutralized) by collisions with other cluster ions, other neutral clusters, residual background gas particles, and thus they may fragment or may be induced to fragment into smaller cluster ions or into monomer ions and/or into neutralized smaller clusters and neutralized monomers, but the resulting cluster ions, neutral clusters, and monomer ions and neutral monomers tend to retain the relatively high velocities and energies that result from having been accelerated through large electric potential differences, with the energy being distributed over the fragments.

As used herein, the terms “GCIB”, “gas cluster ion beam” and “gas cluster ion” are intended to encompass not only ionized beams and ions, but also accelerated beams and ions that have had all or a portion of their charge states modified (including neutralized) following their acceleration. The terms “GCIB” and “gas cluster ion beam” are intended to encompass all beams that comprise accelerated gas clusters even though they may also comprise non-clustered particles. As used herein, the term “Neutral Beam” is intended to mean a beam of neutral gas clusters and/or neutral monomers derived from an accelerated gas cluster ion beam and wherein the acceleration results from acceleration of a gas cluster ion beam. As used herein, the term “monomer” refers equally to either a single atom or a single molecule. The terms “atom,” “molecule,” and “monomer” may be used interchangeably and all refer to the appropriate monomer that is characteristic of the gas under discussion (either a component of a cluster, a component of a cluster ion, or an atom or molecule). For example, a monatomic gas like argon may be referred to in terms of atoms, molecules, or monomers and each of those terms means a single atom. Likewise, in the case of a diatomic gas like nitrogen, it may be referred to in terms of atoms, molecules, or monomers, each term meaning a diatomic molecule. Furthermore a molecular gas like CO₂, may be referred to in terms of atoms, molecules, or monomers, each term meaning a three atom molecule, and so forth. These conventions are used to simplify generic discussions of gases and gas clusters or gas cluster ions independent of whether they are monatomic, diatomic, or molecular in their gaseous form.

When accelerated gas cluster ions are fully dissociated and neutralized, the resulting neutral monomers will have energies approximately equal to the total energy of the original accelerated gas cluster ion, divided by the number, N_{I}, of monomers that comprised the original gas cluster ion at the time it was accelerated. Such dissociated neutral monomers will have energies on the order of from about 1 eV to tens or even as much as a few thousands of eV, depending on the original accelerated energy of the gas cluster ion and the size of the gas cluster at the time of acceleration.

Gas cluster ion beams are generated and transported for purposes of irradiating a workpiece according to known techniques. Various types of holders are known in the art for holding the object in the path of a beam for irradiation and for manipulating or scanning the object to permit irradiation of a multiplicity of portions of the object. Neutral beams may be generated and transported for purposes of irradiating a workpiece according to techniques taught herein.

The present invention may employ a high beam purity method and system for deriving from an accelerated gas cluster ion beam an accelerated neutral gas cluster and/or preferably monomer beam that can be employed for a variety of types of surface and shallow subsurface materials processing and which is capable, for many applications, of superior performance compared to conventional GCIB processing. It can provide well-focused, accelerated, intense neutral monomer beams with particles having energies in the range of from about 1 eV to as much as a few thousand eV. This is an energy range in which it has been impractical with simple, relatively inexpensive apparatus to form intense neutral beams.

These accelerated Neutral Beams are generated by first forming a conventional accelerated GCIB, then partly or essentially fully dissociating it by methods and operating conditions that do not introduce impurities into the beam, then separating the remaining charged portions of the beam from the neutral portion, and subsequently using the resulting accelerated Neutral Beam for workpiece processing. Depending on the degree of dissociation of the gas cluster ions, the Neutral Beam produced may be a mixture of neutral gas monomers and gas clusters or may essentially consist entirely or almost entirely of neutral gas monomers. It is preferred that the accelerated Neutral Beam is a fully dissociated neutral monomer beam.

An advantage of the Neutral Beams that may be produced by the methods and apparatus of this invention, is that they may be used to process electrically insulating materials or high electrical resistivity materials without producing damage to the material due to charging of the surfaces of such materials by beam transported charges as commonly occurs for all ionized beams including GCIB. For example, in semiconductor and other electronic applications, ions often contribute to damaging or destructive charging of thin dielectric films such as oxides, nitrides, etc. The use of Neutral Beams can enable successful beam processing of polymer, dielectric, and/or other electrically insulating or high resistivity materials, coatings, and films in other applications where ion beams may produce undesired side effects due to surface or other charging effects. Further examples include Neutral Beam processing of glass, polymer, and ceramic bio-culture labware and/or environmental sampling surfaces where such beams may be used to improve surface characteristics like, for example, roughness, smoothness, hydrophilicity, biocompatibility, and for sterilization.

Since the parent GCIB, from which accelerated Neutral Beams may be formed by the methods and apparatus of the invention, comprises ions it is readily accelerated to desired energy and is readily focused using conventional ion beam techniques. Upon subsequent dissociation and separation of the charged ions from the neutral particles, the neutral beam particles tend to retain their focused trajectories and may be transported for extensive distances with good effect.

When neutral gas clusters in a jet are ionized by electron bombardment, they become heated and/or excited. This may result in subsequent evaporation of monomers from the ionized gas cluster, after acceleration, as it travels down the beamline. Additionally, collisions of gas cluster ions with background gas molecules in the ionizer, accelerator and beamline regions, also heat and excite the gas cluster ions and may result in additional subsequent evolution of monomers from the gas cluster ions following acceleration. When these mechanisms for evolution of monomers are induced by electron bombardment and/or collision with background gas molecules (and/or other gas clusters) of the same gas from which the GCIB was formed, no contamination is contributed to the beam by the dissociation processes that results in evolving the monomers.

There are other mechanisms that can be employed for dissociating (or inducing evolution of monomers from) gas cluster ions in a GCIB without introducing contamination into the beam. Some of these mechanisms may also be employed to dissociate neutral gas clusters in a neutral gas cluster beam. One mechanism is laser irradiation of the cluster-ion beam using infra-red or other laser energy. Laser-induced heating of the gas cluster ions in the laser irradiated GCIB results in excitement and/or heating of the gas cluster ions and causes subsequent evolution of monomers from the beam. Another mechanism is passing the beam through a thermally heated tube so that radiant thermal energy photons impact the gas cluster ions in beam. The induced heating of the gas cluster ions by the radiant thermal energy in the tube results in excitement and/or heating of the gas cluster ions and causes subsequent evolution of monomers from the beam. In another mechanism, crossing the gas cluster ion beam by a gas jet of the same gas or mixture as the source gas used in formation of the GCIB (or other non-contaminating gas) results in collisions of monomers of the gas in the gas jet with the gas clusters in the ion beam producing excitement and/or heating of the gas cluster ions in the beam and subsequent evolution of monomers from the excited gas cluster ions. By depending entirely on electron bombardment during initial ionization and/or collisions (with other cluster ions, or with background gas molecules of the same gas(es) as those used to form the GCIB) within the beam and/or laser or thermal radiation and/or crossed jet collisions of non-contaminating gas to produce the GCIB dissociation and/or fragmentation, contamination of the beam by collision with other materials is avoided.

As a neutral gas cluster jet from a nozzle travels through an ionizing region where electrons are directed to ionize the clusters, a cluster may remain un-ionized or may acquire a charge state, q, of one or more charges (by ejection of electrons from the cluster by an incident electron). The ionizer operating conditions influence the likelihood that a gas cluster will take on a particular charge state, with more intense ionizer conditions resulting in greater probability that a higher charge state will be achieved. More intense ionizer conditions resulting in higher ionization efficiency may result from higher electron flux and/or higher (within limits) electron energy. Once the gas cluster has been ionized, it is typically extracted from the ionizer, focused into a beam, and accelerated by falling through an electric field. The amount of acceleration of the gas cluster ion is readily controlled by controlling the magnitude of the accelerating electric field. Typical commercial GCIB processing tools generally provide for the gas cluster ions to be accelerated by an electric field having an adjustable accelerating potential, V_{Acc}, typically of, for example, from about 1 kV to 70 kV (but not limited to that range—V_{Acc} up to 200 kV or even more may be feasible). Thus a singly charged gas cluster ion achieves an energy in the range of from 1 to 70 keV (or more if larger V_{Acc} is used) and a multiply charged (for example, without limitation, charge state, q=3 electronic charges) gas cluster ion achieves an energy in the range of from 3 to 210 keV (or more for higher V_{Acc}). For other gas cluster ion charge states and acceleration potentials, the accelerated energy per cluster is qV_{Acc} eV. From a given ionizer with a given ionization efficiency, gas cluster ions will have a distribution of charge states from zero (not ionized) to a higher number such as for example 6 (or with high ionizer efficiency, even more), and the most probable and mean values of the charge state distribution also increase with increased ionizer efficiency (higher electron flux and/or energy). Higher ionizer efficiency also results in increased numbers of gas cluster ions being formed in the ionizer. In many cases, GCIB processing throughput increases when operating the ionizer at high efficiency results in increased GCIB current. A downside of such operation is that multiple charge states that may occur on intermediate size gas cluster ions can increase crater and/or rough interface formation by those ions, and often such effects may operate counterproductively to the intent of the processing. Thus for many GCIB surface processing recipes, selection of the ionizer operating parameters tends to involve more considerations than just maximizing beam current. In some processes, use of a “pressure cell” (see U.S. Pat. No. 7,060,989, to Swenson et al.) may be employed to permit operating an ionizer at high ionization efficiency while still obtaining acceptable beam processing performance by moderating the beam energy by gas collisions in an elevated pressure “pressure cell.”

With the present invention there is no downside to operating the ionizer at high efficiency—in fact such operation is sometimes preferred. When the ionizer is operated at high efficiency, there may be a wide range of charge states in the gas cluster ions produced by the ionizer. This results in a wide range of velocities in the gas cluster ions in the extraction region between the ionizer and the accelerating electrode, and also in the downstream beam. This may result in an enhanced frequency of collisions between and among gas cluster ions in the beam that generally results in a higher degree of fragmentation of the largest gas cluster ions. Such fragmentation may result in a redistribution of the cluster sizes in the beam, skewing it toward the smaller cluster sizes. These cluster fragments retain energy in proportion to their new size (N) and so become less energetic while essentially retaining the accelerated velocity of the initial unfragmented gas cluster ion. The change of energy with retention of velocity following collisions has been experimentally verified (as for example reported in Toyoda, N. et al., “Cluster size dependence on energy and velocity distributions of gas cluster ions after collisions with residual gas,” Nucl. Instr. & Meth. in Phys. Research B 257 (2007), pp 662-665). Fragmentation may also result in redistribution of charges in the cluster fragments. Some uncharged fragments likely result and multi-charged gas cluster ions may fragment into several charged gas cluster ions and perhaps some uncharged fragments. It is understood by the inventors that design of the focusing fields in the ionizer and the extraction region may enhance the focusing of the smaller gas cluster ions and monomer ions to increase the likelihood of collision with larger gas cluster ions in the beam extraction region and in the downstream beam, thus contributing to the dissociation and/or fragmenting of the gas cluster ions.

In an embodiment of the present invention, background gas pressure in the ionizer, acceleration region, and beamline may optionally be arranged to have a higher pressure than is normally utilized for good GCIB transmission. This can result in additional evolution of monomers from gas cluster ions (beyond that resulting from the heating and/or excitement resulting from the initial gas cluster ionization event). Pressure may be arranged so that gas cluster ions have a short enough mean-free-path and a long enough flight path between ionizer and workpiece that they must undergo multiple collisions with background gas molecules.

For a homogeneous gas cluster ion containing N monomers and having a charge state of q and which has been accelerated through an electric field potential drop of V_{Acc} volts, the cluster will have an energy of approximately qV_{Acc}/N_{I} eV per monomer, where N_{I} is the number of monomers in the cluster ion at the time of acceleration. Except for the smallest gas cluster ions, a collision of such an ion with a background gas monomer of the same gas as the cluster source gas will result in additional deposition of approximately qV_{Acc}/N_{I} eV into the gas cluster ion. This energy is relatively small compared to the overall gas cluster ion energy (qV_{Acc}) and generally results in excitation or heating of the cluster and in subsequent evolution of monomers from the cluster. It is believed that such collisions of larger clusters with background gas seldom fragment the cluster but rather heats and/or excites it to result in evolution of monomers by evaporation or similar mechanisms. Regardless of the source of the excitation that results in the evolution of a monomer or monomers from a gas cluster ion, the evolved monomer(s) have approximately the same energy per particle, qV_{Acc}/N_{I} eV, and retain approximately the same velocity and trajectory as the gas cluster ion from which they have evolved. When such monomer evolutions occur from a gas cluster ion, whether they result from excitation or heating due to the original ionization event, a collision, or radiant heating, the charge has a high probability of remaining with the larger residual gas cluster ion. Thus after a sequence of monomer evolutions, a large gas cluster ion may be reduced to a cloud of co-traveling monomers with perhaps a smaller residual gas cluster ion (or possibly several if fragmentation has also occurred). The co-traveling monomers following the original beam trajectory all have approximately the same velocity as that of the original gas cluster ion and each has energy of approximately qV_{Acc}/N_{I} eV. For small gas cluster ions, the energy of collision with a background gas monomer is likely to completely and violently dissociate the small gas cluster and it is uncertain whether in such cases the resulting monomers continue to travel with the beam or are ejected from the beam.

Prior to the GCIB reaching the workpiece, the remaining charged particles (gas cluster ions, particularly small and intermediate size gas cluster ions and some charged monomers, but also including any remaining large gas cluster ions) in the beam are separated from the neutral portion of the beam, leaving only a Neutral Beam for processing the workpiece.

In typical operation, a ratio of power in the neutral beam components to the power in the full (charged plus neutral) beam delivered at the processing target is in the range of from about 5% to 95%, so by the methods and apparatus of the present invention it is possible to convert the corresponding fraction of the kinetic energy of the full accelerated charged beam to that of a Neutral Beam.

The dissociation of the gas cluster ions and thus the production of high neutral monomer beam energy is facilitated by 1) Operating at higher acceleration voltages. This increases qV_{Acc}/N for any given cluster size. 2) Operating at high ionizer efficiency. This increases qV_{Acc}/N for any given cluster size by increasing q and increases cluster-ion on cluster-ion collisions in the extraction region due to the differences in charge states between clusters; 3) Operating at a high ionizer, acceleration region, or beamline pressure or operating with a gas jet crossing the beam, or with a longer beam path, all of which increase the probability of background gas collisions for a gas cluster ion of any given size; 4) Operating with laser irradiation or thermal radiant heating of the beam, which directly promote evolution of monomers from the gas cluster ions; and 5) Operating at higher nozzle gas flow, which increases transport of gas, clustered and perhaps unclustered into the GCIB trajectory, which increases collisions resulting in greater evolution of monomers.

Measurement of the Neutral Beam cannot be made by current measurement as is convenient for gas cluster ion beams. A Neutral Beam power sensor is used to facilitate dosimetry when irradiating a workpiece with a Neutral Beam. The Neutral Beam sensor is a thermal sensor that intercepts the beam (or optionally a known sample of the beam). The rate of rise of temperature of the sensor is related to the energy flux resulting from energetic beam irradiation of the sensor. The thermal measurements must be made over a limited range of temperatures of the sensor to avoid errors due to thermal re-radiation of the energy incident on the sensor. For a GCIB process, the beam power (watts) is equal to the beam current (amps) times V_{Acc}, the beam acceleration voltage. When a GCIB irradiates a workpiece for a period of time (seconds), the energy (joules) received by the workpiece is the product of the beam power and the irradiation time. The processing effect of such a beam when it processes an extended area is distributed over the area (for example, cm²). For ion beams, it has been conveniently conventional to specify a processing dose in terms of irradiated ions/cm², where the ions are either known or assumed to have at the time of acceleration an average charge state, q, and to have been accelerated through a potential difference of, V_{Acc} volts, so that each ion carries an energy of q V_{Acc} eV (an eV is approximately 1.6×10⁻¹⁹ joule). Thus an ion beam dose for an average charge state, q, accelerated by V_{Acc} and specified in ions/cm² corresponds to a readily calculated energy dose expressible in joules/cm². For an accelerated Neutral Beam derived from an accelerated GCIB as utilized in the present invention, the value of q at the time of acceleration and the value of V_{Acc} is the same for both of the (later- formed and separated) charged and uncharged fractions of the beam. The power in the two (neutral and charged) fractions of the GCIB divides proportional to the mass in each beam fraction. Thus for the accelerated Neutral Beam as employed in the invention, when equal areas are irradiated for equal times, the energy dose (joules/cm²) deposited by the Neutral Beam is necessarily less than the energy dose deposited by the full GCIB. By using a thermal sensor to measure the power in the full GCIB P_{G} and that in the Neutral Beam P_{N} (which is commonly found to be about 5% to 95% that of the full GCIB) it is possible to calculate a compensation factor for use in the Neutral Beam processing dosimetry. When P_{N} is aP_{G}, then the compensation factor is, k=1/a. Thus if a workpiece is processed using a Neutral Beam derived from a GCIB, for a time duration is made to be k times greater than the processing duration for the full GCIB (including charged and neutral beam portions) required to achieve a dose of D ions/cm², then the energy doses deposited in the workpiece by both the Neutral Beam and the full GCIB are the same (though the results may be different due to qualitative differences in the processing effects due to differences of particle sizes in the two beams.) As used herein, a Neutral Beam process dose compensated in this way is sometimes described as having an energy/cm² equivalence of a dose of D ions/cm².

Use of a Neutral Beam derived from a gas cluster ion beam in combination with a thermal power sensor for dosimetry in many cases has advantages compared with the use of the full gas cluster ion beam or an intercepted or diverted portion, which inevitably comprises a mixture of gas cluster ions and neutral gas clusters and/or neutral monomers, and which is conventionally measured for dosimetry purposes by using a beam current measurement. Some advantages are as follows:

1) The dosimetry can be more precise with the Neutral Beam using a thermal sensor for dosimetry because the total power of the beam is measured. With a GCIB employing the traditional beam current measurement for dosimetry, only the contribution of the ionized portion of the beam is measured and employed for dosimetry. Minute-to-minute and setup-to-setup changes to operating conditions of the GCIB apparatus may result in variations in the fraction of neutral monomers and neutral clusters in the GCIB. These variations can result in process variations that may be less controlled when the dosimetry is done by beam current measurement.

2) With a Neutral Beam, any material may be processed, including highly insulating materials and other materials that may be damaged by electrical charging effects, without the necessity of providing a source of target neutralizing electrons to prevent workpiece charging due to charge transported to the workpiece by an ionized beam. When employed with conventional GCIB, target neutralization to reduce charging is seldom perfect, and the neutralizing electron source itself often introduces problems such as workpiece heating, contamination from evaporation or sputtering in the electron source, etc. Since a Neutral Beam does not transport charge to the workpiece, such problems are reduced.

3) There is no necessity for an additional device such as a large aperture high strength magnet to separate energetic monomer ions from the Neutral Beam. In the case of conventional GCIB the risk of energetic monomer ions (and other small cluster ions) being transported to the workpiece, where they penetrate producing deep damage, is significant and an expensive magnetic filter is routinely required to separate such particles from the beam. In the case of the Neutral Beam apparatus of the invention, the separation of all ions from the beam to produce the Neutral Beam inherently removes all monomer ions.

One embodiment of the present invention provides a method for sterilizing a workpiece, comprising the steps of: providing a reduced pressure chamber; forming gas-cluster ion-beam comprising gas cluster ions in the reduced pressure chamber; accelerating the gas cluster ions; providing conditions that permit or cause at least partial fragmentation or dissociation of at least part of the accelerated gas cluster ions in the gas cluster ion beam; removing charged particles from the gas cluster ion beam containing at least partially fragmented or dissociated gas cluster ion beam to form an accelerated Neutral Beam having a path in the reduced pressure chamber; providing a workpiece holder in the reduced pressure chamber for holding the workpiece in the Neutral Beam path; irradiating at least a portion of a surface of the workpiece with the accelerated Neutral Beam for sterilizing the portion.

The forming step may include accelerating the gas-cluster ion-beam using an acceleration potential of at least 2 kV. The forming step may comprise forming a gas-cluster ion-beam comprising a noble gas or a mixture of a noble gas with oxygen. The at least a portion of a surface may be an entire surface. The step of providing a workpiece holder may further comprise sterilizing the workpiece holder.

Another embodiment of the present invention provides a method for sterilizing a workpiece, comprising the steps of: a. providing a chamber having an interior and a workpiece holder in the interior; b. initially sterilizing the workpiece holder and the interior of the chamber; c. loading a workpiece onto the workpiece holder to be held thereby for sterilization; d. reducing the pressure in the chamber; e. forming an accelerated gas-cluster ion-beam; f. providing conditions that permit or cause at least partial fragmentation or dissociation of the accelerated gas cluster ion beam; g. removing charged particles from the at least partially fragmented or dissociated accelerated gas cluster ion beam to form an accelerated Neutral Beam; h. directing the accelerated Neutral Beam onto the workpiece; i. irradiating at least a portion of a surface of the workpiece with the accelerated neutral beam; j. discontinuing irradiation when the at least a portion of a surface of the workpiece has received a predetermined dose; and k. unloading the workpiece from the workpiece holder and removing it from the chamber.

The method may further comprise the step of venting the chamber with a sterile gas. The at least a portion of a surface may be an entire surface. The step of unloading may include placing the workpiece directly into a sterile container.

For a better understanding of the present invention, together with other and further objects thereof, reference is made to the accompanying drawings and detailed description and in the appended claims.

### BRIEF DESCRIPTION OF THE DRAWINGS

FIG. 1 is a is a schematic view of a GCIB processing system of the present invention;

FIG. 2 is an enlarged view of a portion of the GCIB processing system, showing the workpiece holder and manipulator for handling the object to be sterilized;

FIG. 3 is a schematic illustrating elements of a GCIB processing apparatus 1100 for processing a workpiece using a GCIB;

FIG. 4 is a schematic illustrating elements of another GCIB processing apparatus 1200 for workpiece processing using a GCIB, wherein scanning of the ion beam and manipulation of the workpiece is employed;

FIG. 5 is a schematic of a Neutral Beam processing apparatus 1300 according to an embodiment of the invention, which uses electrostatic deflection plates to separate the charged and uncharged beams;

FIG. 6 is a schematic of a Neutral Beam processing apparatus 1400 according to an embodiment of the invention, using a thermal sensor for Neutral Beam measurement;

FIG. 7 is a schematic of a sterilizing system for GCIB sterilization of workpieces;

FIG. 8A is a photograph of a control titanium foil showing bacterial colonies growing thereon;

FIG. 8B is a photograph of a conventionally sterilized titanium foil showing no bacterial colonies growing thereon; and

FIG. 8C is a photograph of a GCIB irradiated titanium foil showing no bacterial colonies growing thereon, indicating effectiveness of GCIB sterilization.

### DETAILED DESCRIPTION OF THE PREFERRED METHODS AND EMBODIMENTS

In the following description, for simplification, item numbers from earlier-described figures may appear in subsequently-described figures without discussion. Likewise, items discussed in relation to earlier figures may appear in subsequent figures without item numbers or additional description. In such cases items with like numbers are like items and have the previously-described features and functions, and illustration of items without item numbers shown in the present figure refer to like items having the same functions as the like items illustrated in earlier-discussed numbered figures.

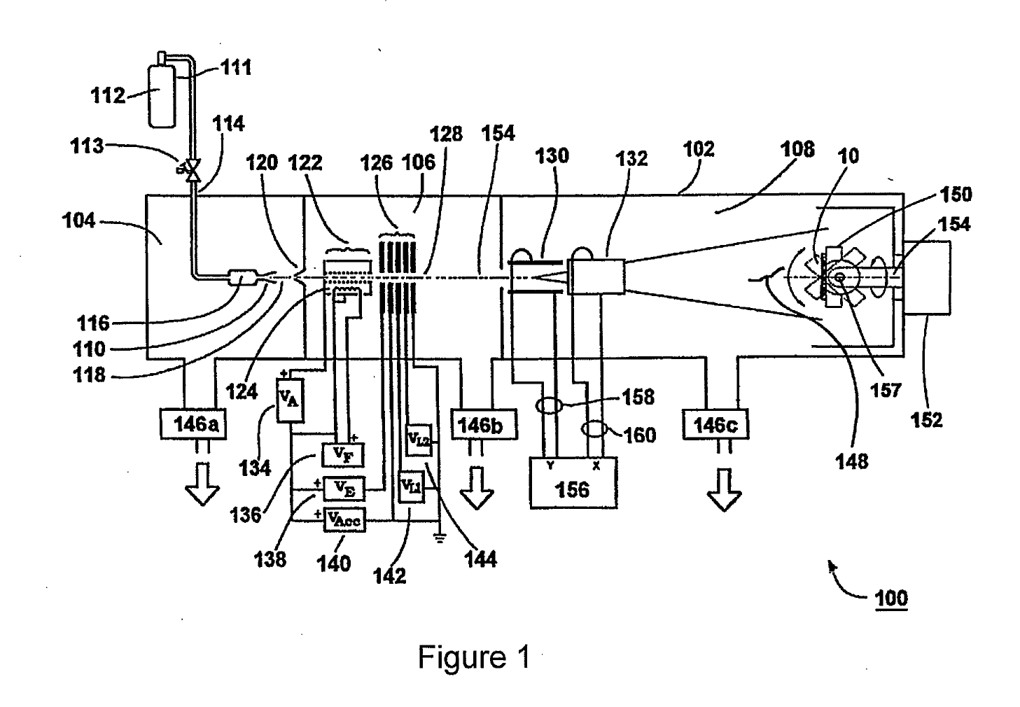

FIG. 1 shows an embodiment of the (GCIB) processor 100 of this invention utilized for the surface sterilization of a workpiece 10 (which may be a medical device, surgical implant, or medical prosthesis or some other sterilizable object). Although not limited to the specific components described herein, the GCIB processor 100 is made up of a vacuum vessel 102 which is divided into three communicating chambers, a source chamber 104, an ionization/acceleration chamber 106, and a process chamber 108 which includes therein a uniquely designed workpiece holder 150 capable of positioning the medical device for uniform processing by a gas-cluster ion-beam.

During the processing method of this invention, the three chambers are evacuated to suitable operating pressures by vacuum pumping systems 146a, 146b, and 146c, respectively. A condensable source gas 112 (for example argon, O₂, etc.) stored in a cylinder 111 is admitted under pressure through gas metering valve 113 and gas feed tube 114 into stagnation chamber 116 and is ejected into the substantially lower pressure vacuum through a properly shaped nozzle 110, resulting in a supersonic gas jet 118. Cooling, which results from the expansion of the jet, causes a portion of the gas jet 118 to condense into clusters, each consisting of from several to several thousand weakly bound atoms or molecules, and typically having a distribution with a most likely size of hundreds to thousands of atoms or molecules. A gas skimmer aperture 120 partially separates the gas molecules that have not condensed into a cluster jet from the cluster jet so as to minimize pressure in the downstream regions where such higher pressures would be detrimental (e.g., ionizer 122, high voltage electrodes 126, and process chamber 108). Suitable condensable source gases 112 include, but are not necessarily limited to argon or other noble gases, oxygen, oxygen-containing gases, other reactive gases, and mixtures of these or other gases.

After the supersonic gas jet 118 containing gas clusters has been formed, the clusters are ionized in an ionizer 122. The ionizer 122 is typically an electron impact ionizer that produces thermoelectrons from one or more incandescent filament(s) 124 and accelerates and directs the electrons causing them to collide with the gas clusters in the gas jet 118, where the jet passes through the ionizer 122. The electron impact ejects electrons from the clusters, causing a portion the clusters to become positively ionized. A set of suitably biased high voltage electrodes 126 extracts the cluster ions from the ionizer 122, forming a beam, then accelerates the cluster ions to a desired energy (typically using an acceleration potential of from about 2 keV to as much as 100 keV) and focuses them to form a GCIB 128 having an initial trajectory 154. Filament power supply 136 provides voltage V_{F} to heat the ionizer filament 124. Anode power supply 134 provides voltage V_{A} to accelerate thermoelectrons emitted from filament 124 to cause them to bombard the cluster containing gas jet 118 to produce ions. Extraction power supply 138 provides voltage V_{E} to bias a high voltage electrode to extract ions from the ionizing region of ionizer 122 and to form a GCIB 128. Accelerator power supply 140 provides voltage V_{Acc} to bias a high voltage electrode with respect to the ionizer 122 so as to result in a total GCIB acceleration potential equal to V_{Acc} volts. One or more lens power supplies (142 and 144, for example) may be provided to bias high voltage electrodes with potentials (V_{L1} and V_{L2}, for example) to focus the GCIB 128.

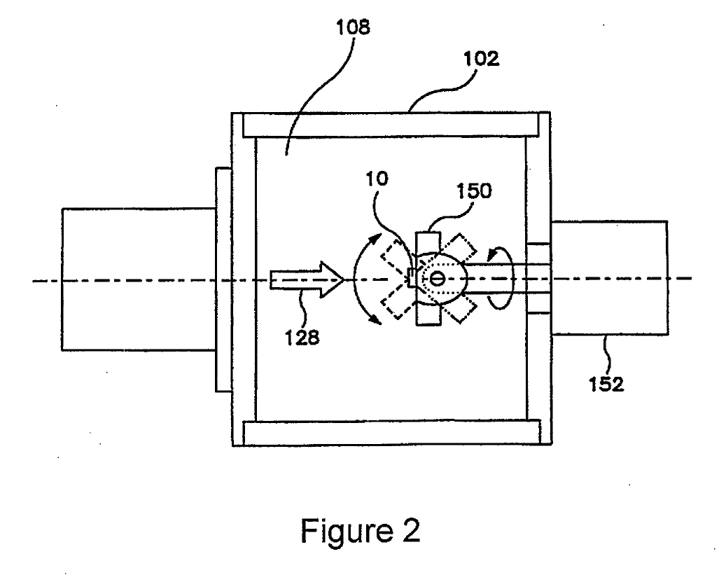

Referring now to FIG. 2, a workpiece 10 to be processed by GCIB irradiation using the GCIB processor 100 is/are held on a workpiece holder 150, disposed in the path of the GCIB 128. In order to facilitate uniform processing of one or more surfaces or surface regions of the workpiece 10, the workpiece holder 150 is designed in a manner set forth below to position and/or manipulate the workpiece 10 to expose multiple surface regions for GCIB processing.

As will be explained further hereinbelow, the practice of the present invention is facilitated by an ability to control positioning of the object to be sterilized with respect to the GCIB is required to assure irradiation of all necessary surfaces of the object being sterilized. Objects being sterilized may have multiple surfaces with different surface orientations. It is desirable that there be a capability for positioning and orientating the object to be sterilized with respect to the GCIB. This requires a fixture or workpiece holder 150 with the ability to be fully articulated in order to orient all desired surfaces of a workpiece 10 to be sterilized, within the GCIB to assure incidence for the desired surface irradiation effect. More specifically, when processing a workpiece 10, the workpiece holder 150 is rotated and articulated by an articulation/rotation mechanism 152 located at the end of the GCIB processor 100.

Referring again to FIG. 1, the articulation/rotation mechanism 152 preferably permits 360 degrees of device rotation about longitudinal axis coinciding with the trajectory 154 and sufficient device articulation about an axis 157 that may be perpendicular to the longitudinal axis coinciding with the trajectory 154 to expose the objects surfaces to the GCIB for irradiation. Under certain conditions, depending upon the size of the workpiece 10, which is to be sterilized, a scanning system may be desirable to produce uniform irradiation of the medical device with the GCIB 128. Although not necessary for all GCIB processing, two pairs of orthogonally oriented electrostatic scan plates 130 and 132 may be utilized to produce a raster or other beam scanning pattern over an extended processing area. When such beam scanning is performed, a scan generator 156 provides X-axis and Y-axis scanning signal voltages to the pairs of scan plates 130 and 132 through lead pairs 158 and 160 respectively. The scanning signal voltages may be triangular waves of different frequencies that cause the GCIB 128 to be converted into a scanned GCIB 148, which scans an entire surface or extended region of the workpiece 10. As an alternative to scanning the GCIB across the workpiece 10, the workpiece holder 150 may be designed to move the medical device through a stationary GCIB in a scanning motion relative to the GCIB.

When beam scanning over an extended region is not desired, processing is generally confined to a region that is defined by the diameter of the beam. The diameter of the beam at the surface of the workpiece 10 can be set by selecting the voltages (V_{L1} and/or V_{L2}) of one or more lens power supplies (142 and 144 shown for example) to provide the desired beam diameter at the workpiece.

Gas-cluster ion-beam processing is used in semiconductor processing and fabrication as a technology that provides extreme processing accuracy. A further advantage to GCIB sterilization over other radiation techniques is the unique ability to process only the exposed surface while not having any effect on the sub-surface regions of the product. GCIB does not significantly penetrate nor permeate the object being sterilized and has no effect on the bulk portion of the object. In an embodiment of the invention, a Neutral Beam derived from an accelerated gas cluster ion beam is employed to sterilize insulating (and other sensitive) surfaces.

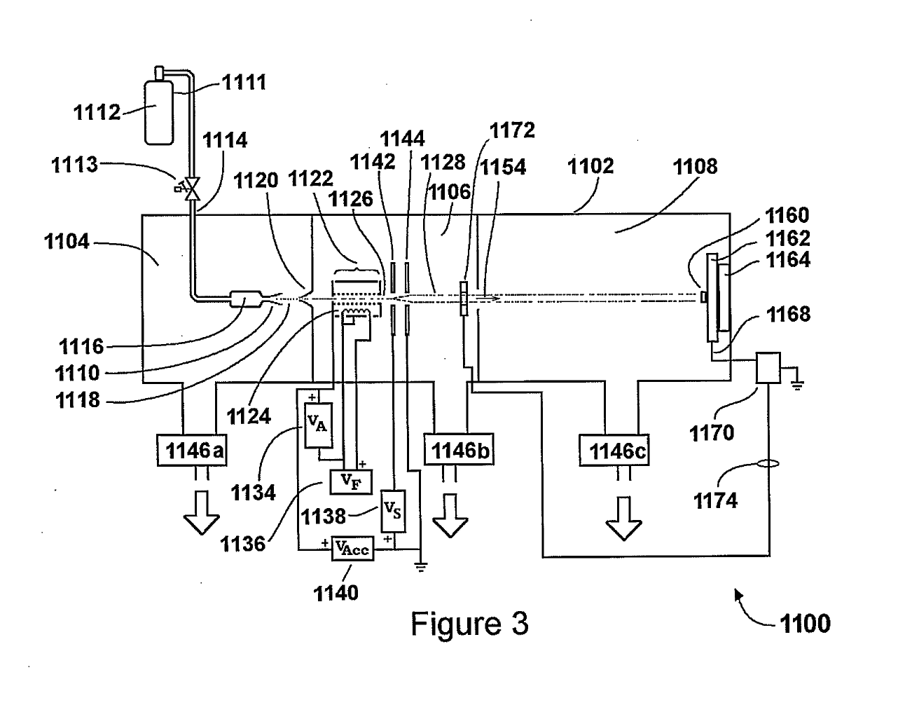

Reference is now made to FIG. 3, which shows a schematic configuration for a GCIB processing apparatus 1100. A low-pressure vessel 1102 has three fluidly connected chambers: a nozzle chamber 1104, an ionization/acceleration chamber 1106, and a processing chamber 1108. The three chambers are evacuated by vacuum pumps 1146a, 1146b, and 1146c, respectively. A pressurized condensable source gas 1112 (for example argon) stored in a gas storage cylinder 1111 flows through a gas metering valve 1113 and a feed tube 1114 into a stagnation chamber 1116. Pressure (typically a few atmospheres) in the stagnation chamber 1116 results in ejection of gas into the substantially lower pressure vacuum through a nozzle 1110, resulting in formation of a supersonic gas jet 1118. Cooling, resulting from the expansion in the jet, causes a portion of the gas jet 1118 to condense into clusters, each consisting of from several to several thousand weakly bound atoms or molecules. A gas skimmer aperture 1120 is employed to control flow of gas into the downstream chambers by partially separating gas molecules that have not condensed into a cluster jet from the cluster jet. Excessive pressure in the downstream chambers can be detrimental by interfering with the transport of gas cluster ions and by interfering with management of the high voltages that may be employed for beam formation and transport. Suitable condensable source gases 1112 include, but are not limited to argon and other condensable noble gases, nitrogen, carbon dioxide, oxygen, and many other gases and/or gas mixtures. After formation of the gas clusters in the supersonic gas jet 1118, at least a portion of the gas clusters are ionized in an ionizer 1122 that is typically an electron impact ionizer that produces electrons by thermal emission from one or more incandescent filaments 1124 (or from other suitable electron sources) and accelerates and directs the electrons, enabling them to collide with gas clusters in the gas jet 1118. Electron impacts with gas clusters eject electrons from some portion of the gas clusters, causing those clusters to become positively ionized. Some clusters may have more than one electron ejected and may become multiply ionized. Control of the number of electrons and their energies after acceleration typically influences the number of ionizations that may occur and the ratio between multiple and single ionizations of the gas clusters. A suppressor electrode 1142, and grounded electrode 1144 extract the cluster ions from the ionizer exit aperture 1126, accelerate them to a desired energy (typically with acceleration potentials of from several hundred V to several tens of kV), and focuses them to form a GCIB 1128. The region that the GCIB 1128 traverses between the ionizer exit aperture 126 and the suppressor electrode 1142 is referred to as the extraction region. The axis (determined at the nozzle 1110), of the supersonic gas jet 1118 containing gas clusters is substantially the same as the axis 1154 of the GCIB 1128. Filament power supply 1136 provides filament voltage V_{f} to heat the ionizer filament 1124. Anode power supply 1134 provides anode voltage V_{A} to accelerate thermoelectrons emitted from filament 1124 to cause the thermoelectrons to irradiate the cluster-containing gas jet 1118 to produce cluster ions. A suppression power supply 1138 supplies suppression voltage V_{S} (on the order of several hundred to a few thousand volts) to bias suppressor electrode 1142. Accelerator power supply 1140 supplies acceleration voltage V_{Acc} to bias the ionizer 1122 with respect to suppressor electrode 1142 and grounded electrode 1144 so as to result in a total GCIB acceleration potential equal to V_{Acc}. Suppressor electrode 1142 serves to extract ions from the ionizer exit aperture 1126 of ionizer 1122 and to prevent undesired electrons from entering the ionizer 1122 from downstream, and to form a focused GCIB 1128.

A workpiece 1160, which may (for example) be a medical device, a semiconductor material, an optical element, or other workpiece to be processed by GCIB processing, is held on a workpiece holder 1162, which disposes the workpiece in the path of the GCIB 1128. The workpiece holder is attached to but electrically insulated from the processing chamber 1108 by an electrical insulator 1164. Thus, GCIB 1128 striking the workpiece 1160 and the workpiece holder 1162 flows through an electrical lead 1168 to a dose processor 1170. A beam gate 1172 controls transmission of the GCIB 1128 along axis 1154 to the workpiece 1160. The beam gate 1172 typically has an open state and as closed state that is controlled by a linkage 1174 that may be (for example) electrical, mechanical, or electromechanical. Dose processor 1170 controls the open/closed state of the beam gate 1172 to manage the GCIB dose received by the workpiece 1160 and the workpiece holder 1162. In operation, the dose processor 1170 opens the beam gate 1172 to initiate GCIB irradiation of the workpiece 1160. Dose processor 1170 typically integrates GCIB electrical current arriving at the workpiece 1160 and workpiece holder 1162 to calculate an accumulated GCIB irradiation dose. At a predetermined dose, the dose processor 1170 closes the beam gate 1172, terminating processing when the predetermined dose has been achieved.

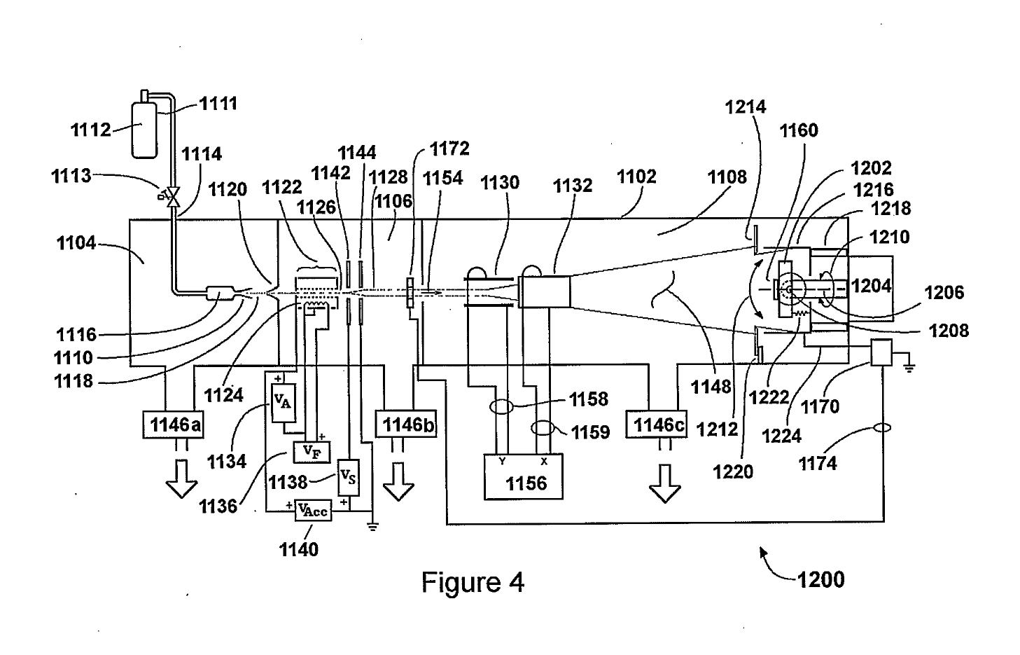

FIG. 4 shows a schematic illustrating elements of another GCIB processing apparatus 1200 for workpiece processing using a GCIB, wherein scanning of the ion beam and manipulation of the workpiece is employed. A workpiece 1160 to be processed by the GCIB processing apparatus 1200 is held on a workpiece holder 1202, disposed in the path of the GCIB 1128. In order to accomplish uniform processing of the workpiece 1160, the workpiece holder 1202 is designed to manipulate workpiece 1160, as may be required for uniform processing.

Any workpiece surfaces that are non-planar, for example, spherical or cup-like, rounded, irregular, or other un-flat configuration, may be oriented within a range of angles with respect to the beam incidence to obtain optimal GCIB processing of the workpiece surfaces. The workpiece holder 1202 can be fully articulated for orienting all non-planar surfaces to be processed in suitable alignment with the GCIB 1128 to provide processing optimization and uniformity. More specifically, when the workpiece 1160 being processed is non-planar, the workpiece holder 1202 may be rotated in a rotary motion 1210 and articulated in articulation motion 1212 by an articulation/rotation mechanism 1204. The articulation/rotation mechanism 1204 may permit 360 degrees of device rotation about longitudinal axis 1206 (which is coaxial with the axis 1154 of the GCIB 1128) and sufficient articulation about an axis 1208 perpendicular to axis 1206 to maintain the workpiece surface to within a desired range of beam incidence.

Under certain conditions, depending upon the size of the workpiece 1160, a scanning system may be desirable to produce uniform irradiation of a large workpiece. Although often not necessary for GCIB processing, two pairs of orthogonally oriented electrostatic scan plates 1130 and 1132 may be utilized to produce a raster or other scanning pattern over an extended processing area. When such beam scanning is performed, a scan generator 1156 provides X-axis scanning signal voltages to the pair of scan plates 1132 through lead pair 1159 and Y-axis scanning signal voltages to the pair of scan plates 1130 through lead pair 1158. The scanning signal voltages are commonly triangular waves of different frequencies that cause the GCIB 1128 to be converted into a scanned GCIB 1148, which scans the entire surface of the workpiece 1160. A scanned beam-defining aperture 1214 defines a scanned area. The scanned beam-defining aperture 1214 is electrically conductive and is electrically connected to the low-pressure vessel 1102 wall and supported by support member 1220. The workpiece holder 1202 is electrically connected via a flexible electrical lead 1222 to a faraday cup 1216 that surrounds the workpiece 1160 and the workpiece holder 1202 and collects all the current passing through the defining aperture 1214. The workpiece holder 1202 is electrically isolated from the articulation/rotation mechanism 1204 and the faraday cup 1216 is electrically isolated from and mounted to the low-pressure vessel 1102 by insulators 1218. Accordingly, all current from the scanned GCIB 1148, which passes through the scanned beam-defining aperture 1214 is collected in the faraday cup 1216 and flows through electrical lead 1224 to the dose processor 1170. In operation, the dose processor 1170 opens the beam gate 1172 to initiate GCIB irradiation of the workpiece 1160. The dose processor 1170 typically integrates GCIB electrical current arriving at the workpiece 1160 and workpiece holder 1202 and faraday cup 1216 to calculate an accumulated GCIB irradiation dose per unit area. At a predetermined dose, the dose processor 1170 closes the beam gate 1172, terminating processing when the predetermined dose has been achieved. During the accumulation of the predetermined dose, the workpiece 1160 may be manipulated by the articulation/rotation mechanism 1204 to ensure processing of all desired surfaces.

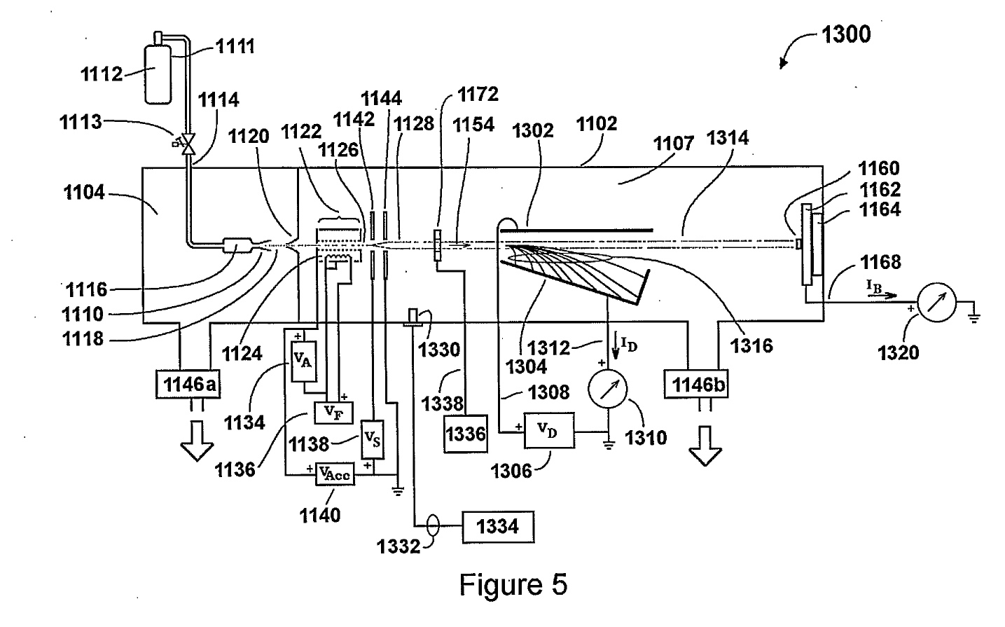

FIG. 5 is a schematic of a Neutral Beam processing apparatus 1300 of an exemplary type that may be employed for Neutral Beam processing according to embodiments of the invention. It uses electrostatic deflection plates to separate the charged and uncharged portions of a GCIB. A beamline chamber 1107 encloses the ionizer and accelerator regions and the workpiece processing regions. The beamline chamber 1107 has high conductance and so the pressure is substantially uniform throughout. A vacuum pump 1146b evacuates the beamline chamber 1107. Gas flows into the beamline chamber 1107 in the form of clustered and unclustered gas transported by the gas jet 1118 and in the form of additional unclustered gas that leaks through the gas skimmer aperture 1120. A pressure sensor 1330 transmits pressure data from the beamline chamber 1107 through an electrical cable 1332 to a pressure sensor controller 1334, which measures and displays pressure in the beamline chamber 1107. The pressure in the beamline chamber 1107 depends on the balance of gas flow into the beamline chamber 1107 and the pumping speed of the vacuum pump 1146b. By selection of the diameter of the gas skimmer aperture 1120, the flow of source gas 1112 through the nozzle 1110, and the pumping speed of the vacuum pump 1146b, the pressure in the beamline chamber 1107 equilibrates at a pressure, P_{B}, determined by design and by nozzle flow. The beam flight path from grounded electrode 1144 to workpiece holder 162, is for example, 100 cm. By design and adjustment P_{B} may be approximately 6×10⁻⁵ torr (8×10⁻³ pascal). Thus the product of pressure and beam path length is approximately 6×10⁻³ torr-cm (0.8 pascal-cm) and the gas target thickness for the beam is approximately 1.94×10¹⁴ gas molecules per cm², which is observed to be effective for dissociating the gas cluster ions in the GCIB 1128. V_{Acc} may be for example 30 kV and the GCIB 1128 is accelerated by that potential. A pair of deflection plates (1302 and 1304) is disposed about the axis 1154 of the GCIB 1128. A deflector power supply 1306 provides a positive deflection voltage V_{D} to deflection plate 1302 via electrical lead 1308. Deflection plate 1304 is connected to electrical ground by electrical lead 1312 and through current sensor/display 1310. Deflector power supply 1306 is manually controllable. V_{D} may be adjusted from zero to a voltage sufficient to completely deflect the ionized portion 1316 of the GCIB 1128 onto the deflection plate 1304 (for example a few thousand volts). When the ionized portion 1316 of the GCIB 1128 is deflected onto the deflection plate 1304, the resulting current, I_{D} flows through electrical lead 1312 and current sensor/display 1310 for indication. When V_{D} is zero, the GCIB 1128 is undeflected and travels to the workpiece 1160 and the workpiece holder 1162. The GCIB beam current I_{B} is collected on the workpiece 1160 and the workpiece holder 1162 and flows through electrical lead 1168 and current sensor/display 1320 to electrical ground. I_{B} is indicated on the current sensor/display 1320. A beam gate 1172 is controlled through a linkage 1338 by beam gate controller 1336. Beam gate controller 1336 may be manual or may be electrically or mechanically timed by a preset value to open the beam gate 1172 for a predetermined interval. In use, V_{D} is set to zero, the beam current, I_{B}, striking the workpiece holder is measured. Based on previous experience for a given GCIB process recipe, an initial irradiation time for a given process is determined based on the measured current, I_{B}. V_{D} is increased until all measured beam current is transferred from I_{B} to I_{D} and I_{D} no longer increases with increasing V_{D}. At this point a Neutral Beam 1314 comprising energetic dissociated components of the initial GCIB 1128 irradiates the workpiece holder 1162. The beam gate 1172 is then closed and the workpiece 1160 placed onto the workpiece holder 1162 by conventional workpiece loading means (not shown). The beam gate 1172 is opened for the predetermined initial radiation time. After the irradiation interval, the workpiece may be examined and the processing time adjusted as necessary to calibrate the duration of Neutral Beam processing based on the measured GCIB beam current I_{B}. Following such a calibration process, additional workpieces may be processed using the calibrated exposure duration.

The Neutral Beam 1314 contains a repeatable fraction of the initial energy of the accelerated GCIB 1128. The remaining ionized portion 1316 of the original GCIB 1128 has been removed from the Neutral Beam 1314 and is collected by the grounded deflection plate 1304. The ionized portion 1316 that is removed from the Neutral Beam 1314 may include monomer ions and gas cluster ions including intermediate size gas cluster ions. Because of the monomer evaporation mechanisms due to cluster heating during the ionization process, intra-beam collisions, background gas collisions, and other causes (all of which result in erosion of clusters) the Neutral Beam substantially consists of neutral monomers, while the separated charged particles are predominately cluster ions. The inventors have confirmed this by suitable measurements that include re-ionizing the Neutral Beam and measuring the charge to mass ratio of the resulting ions. The separated charged beam components largely consist of cluster ions of intermediate size as well as monomer ions and perhaps some large cluster ions. As will be shown below, certain superior process results are obtained by processing workpieces using this Neutral Beam.

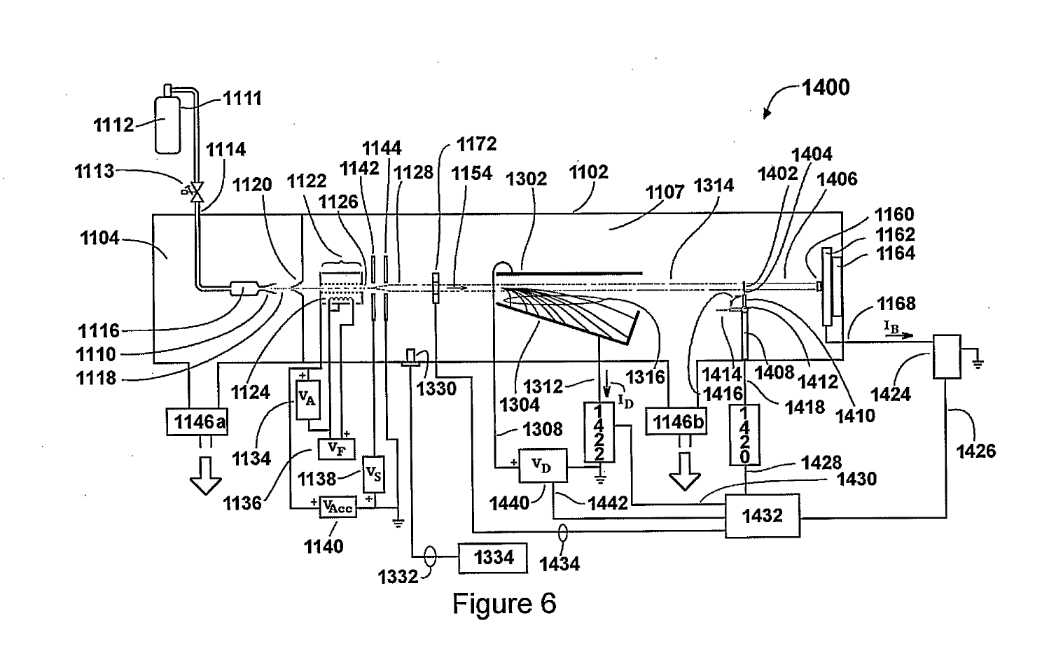

FIG. 6 is a schematic of a Neutral Beam processing apparatus 1400 as may, for example, be used in generating Neutral Beams as may be employed in embodiments of the invention. It uses a thermal sensor for Neutral Beam measurement. A thermal sensor 1402 attaches via low thermal conductivity attachment 1404 to a rotating support arm 1410 attached to a pivot 1412. Actuator 1408 moves thermal sensor 1402 via a reversible rotary motion 1416 between positions that intercept the Neutral Beam 1314 or GCIB 1128 and a parked position indicated by 1414 where the thermal sensor 1402 does not intercept any beam. When thermal sensor 1402 is in the parked position (indicated by 1414) the GCIB 1128 or Neutral Beam 1314 continues along path 1406 for irradiation of the workpiece 1160 and/or workpiece holder 1162. A thermal sensor controller 1420 controls positioning of the thermal sensor 1402 and performs processing of the signal generated by thermal sensor 1402. Thermal sensor 1402 communicates with the thermal sensor controller 1420 through an electrical cable 1418. Thermal sensor controller 1420 communicates with a dosimetry controller 1432 through an electrical cable 1428. A beam current measurement device 1424 measures beam current I_{B} flowing in electrical lead 1168 when the GCIB 1128 strikes the workpiece 1160 and/or the workpiece holder 1162. Beam current measurement device 1424 communicates a beam current measurement signal to dosimetry controller 1432 via electrical cable 1426. Dosimetry controller 1432 controls setting of open and closed states for beam gate 1172 by control signals transmitted via linkage 1434. Dosimetry controller 1432 controls deflector power supply 1440 via electrical cable 1442 and can control the deflection voltage V_{D} between voltages of zero and a positive voltage adequate to completely deflect the ionized portion 1316 of the GCIB 1128 to the deflection plate 1304. When the ionized portion 1316 of the GCIB 1128 strikes deflection plate 1304, the resulting current I_{D} is measured by current sensor 1422 and communicated to the dosimetry controller 1432 via electrical cable 1430. In operation dosimetry controller 1432 sets the thermal sensor 1402 to the parked position 1414, opens beam gate 1172, sets V_{D} to zero so that the full GCIB 1128 strikes the workpiece holder 1162 and/or workpiece 1160. The dosimetry controller 1432 records the beam current I_{B} transmitted from beam current measurement device 1424. The dosimetry controller 1432 then moves the thermal sensor 1402 from the parked position 1414 to intercept the GCIB 1128 by commands relayed through thermal sensor controller 1420. Thermal sensor controller 1420 measures the beam energy flux (power) of GCIB 1128 by calculation based on the heat capacity of the sensor and measured rate of temperature rise of the thermal sensor 1402 as its temperature rises through a predetermined measurement temperature (for example 70 degrees C.) and communicates the calculated beam energy flux to the dosimetry controller 1432 which then calculates a calibration of the beam, energy flux as measured by the thermal sensor 1402 and the corresponding beam current measured by the beam current measurement device 1424. The dosimetry controller 1432 then parks the thermal sensor 1402 at parked position 1414, allowing it to cool and commands application of positive V_{D} to deflection plate 1302 until all of the current I_{D} due to the ionized portion of the GCIB 1128 is transferred to the deflection plate 1304. The current sensor 1422 measures the corresponding I_{D} and communicates it to the dosimetry controller 1432. The dosimetry controller also moves the thermal sensor 1402 from parked position 1414 to intercept the Neutral Beam 1314 by commands relayed through thermal sensor controller 420. Thermal sensor controller 420 measures the beam energy flux of the Neutral Beam 1314 using the previously determined calibration factor and the rate of temperature rise of the thermal sensor 1402 as its temperature rises through the predetermined measurement temperature and communicates the Neutral Beam energy flux to the dosimetry controller 1432. The dosimetry controller 1432 calculates a neutral beam fraction, which is the ratio of the thermal measurement of the Neutral Beam 1314 energy flux to the thermal measurement of the full GCIB 1128 energy flux. Under typical operation, a Neutral Beam fraction of about 5% to about 95% is achieved. Before beginning processing, the dosimetry controller 1432 also measures the current, I_{D}, and determines a current ratio between the initial values of I_{B} and I_{D}. During processing, the instantaneous I_{D} measurement multiplied by the initial I_{B}/I_{D} ratio may be used as a proxy for continuous measurement of the I_{B} and employed for dosimetry during control of processing by the dosimetry controller 1432. Thus the dosimetry controller 1432 can compensate any beam fluctuation during workpiece processing, just as if an actual beam current measurement for the full GCIB 1128 were available. The dosimetry controller uses the neutral beam fraction to compute a desired processing time for a particular beam process. During the process, the processing time can be adjusted based on the calibrated measurement of I_{D} for correction of any beam fluctuation during the process.

The sterilization process can be described as follows. First, the device to be sterilized is placed into a vacuum vessel mounted on suitable fixtures to allow the device to be manipulated so that all surface areas can be exposed to the GCIB or Neutral Beam during processing. Second, the vessel is pumped to a vacuum condition, ideally at a pressure lower than 1.3×10⁻² pascal. Once process-level vacuum is attained in the vacuum vessel, a gate valve is opened between the processing vacuum vessel and the main GCIB tool. The gas-cluster ion-beam is then allowed to expose all surfaces of the substrate to gas-cluster ion bombardment to a dose equal to or greater than 10¹³ ions per square centimeter, (or in the case of a Neutral Beam, an exposure that yields an equivalent energy/cm² dose) a level sufficient to assure adequate numbers of cluster ion or neutral particle impacts upon every biologically active organism. The gas clusters are typically formed from source gases such as, but are not necessarily limited to, argon or other noble gases, oxygen, oxygen-containing gases, other reactive gases, and mixtures of these or other gases.

Once the clusters are generated and formed into a beam, applying a high voltage accelerating potential of from 5 to 200 kV accelerates them. This high voltage potential accelerates the gas-cluster ions toward the substrate and thereby causes the clusters to impact the surface to be sterilized, releasing all their energy into that surface. In the case of Neutral Beams, the accelerated gas-cluster ions are at least partially dissociated and then the charged particles are separated from the GCIB to leave only the Neutral Beam directed toward the object to be sterilized. The impact and energy release at the point of each cluster impact causes an intense thermal spike exceeding 1000 degrees Kelvin, but of extremely short duration, to occur only in the immediate localized region, typically in the topmost 100 angstroms only. Energetic neutral particles also penetrate shallowly, depositing their energy within the topmost surface layers, creating local heating and structure disruption resulting in sterilization. The high vacuum system pumps away all volatile organics and maintains a contaminant free surface state while processing continues. When the entire surface has been bombarded at the desired dose, the irradiation is terminated. The sterilized piece is now maintained in a high-vacuum contaminant-free state until the vacuum system is closed off and the vessel is returned to atmosphere by backfilling with an inert, sterile gas.

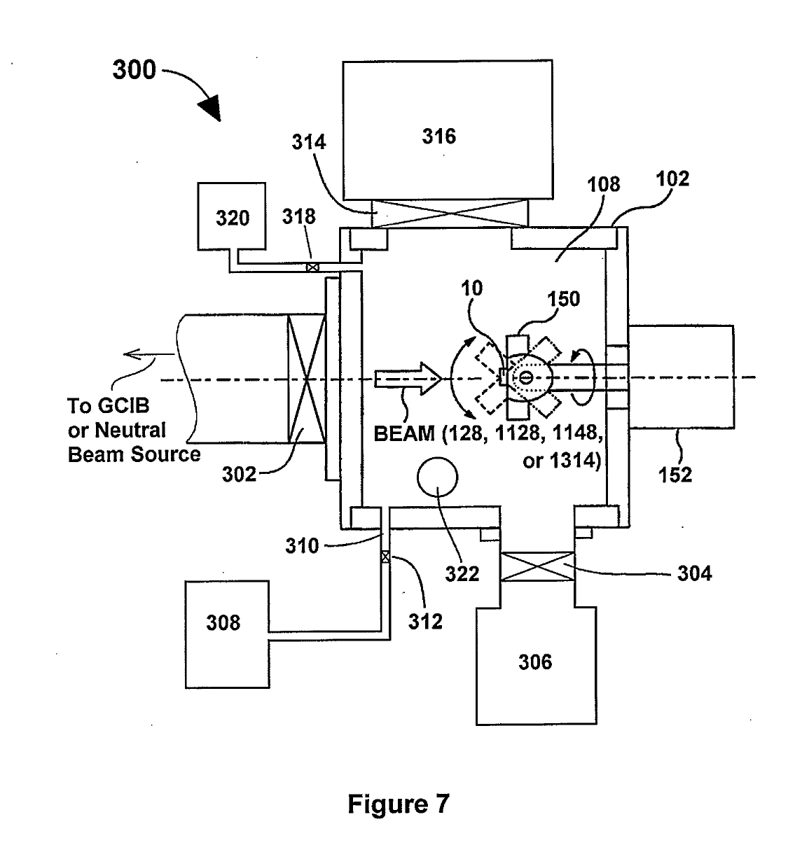

FIG. 7 is a schematic of a sterilizing system 300 specifically adapted according to the invention for GCIB or accelerated Neutral Beam sterilization processes. Such a system may be combined with either a GCIB source or an accelerated Neutral Beam source (derived from a GCIB as disclosed herein.) The vacuum vessel 102 includes a process chamber 108 that can be isolated from the beam source by an isolation valve 302. Isolation valve 302 has open and closed states. In the open state, isolation valve 302 permits a GCIB or Neutral Beam (128, 1128, 1148, or 1314) to enter the process chamber 108 for irradiating a workpiece 10 to be sterilized while held by a workpiece holder 150. The workpiece holder 150 may be designed as previously described (during discussion of FIGS. 1 and 2 above) to rotate and/or articulate the workpiece 10 by means of articulation/rotation mechanism 152, or it may have other designs for fixedly supporting or for manipulating the workpiece 10, as will be readily apparent to those skilled in the art, for exposing single or multiple surfaces of the workpiece to the GCIB or Neutral Beam (128, 1128, 1148, or 1314) (as may be required by the geometry of the workpiece and the sterilization requirements.) In the closed state, isolation valve 302 isolates the process chamber 108 from the beam source. A GCIB source may be similar to that shown in FIG. 1 or FIG. 3 or FIG. 4 or may be some other conventional GCIB source. A Neutral Beam source may be as described in FIG. 5 or FIG. 6 or any system that generates an accelerated GCIB, permits or induces at least partial dissociation of the beam, and then separates charged particles from the beam leaving an accelerated Neutral Beam for the workpiece processing. The GCIB or Neutral Beam (128, 1128, 1148, or 1314) provided by the beam source may be a scanned or an un-scanned GCIB as may be suitable for the size of the workpiece 10 to be sterilized, or it may be a Neutral Beam. When a Neutral Beam is used, it is difficult to scan the beam across the workpiece, but (not shown) the workpiece holder 150 may be designed according to conventional designs to move the workpiece through a stationary Neutral Beam in a scanning motion relative to the Neutral Beam.

A vacuum system 306 is coupled to the process chamber 108 by an isolation valve 304. Isolation valve 304 has open and closed states and may be manually or automatically controlled. When in the open state, isolation valve 304 permits evacuation of the process chamber 108 by the vacuum system 306. When in the closed state, isolation valve 304 inhibits evacuation of the process chamber 108 and permits the introduction of non-vacuum atmospheres to the process chamber 108. A vent line 310 has a valve 312 for controlling introduction of a sterile venting gas 308 to the process chamber 108. A sterilant gas 320 may optionally be introduced to the process chamber 108 through valve 318 for initial sterilization of the process chamber 108 and workpiece holder 150 or for re-sterilization after a contamination event. An optional radiation source 322, which may be a short-wave ultraviolet radiation source may also be used for initial sterilization of the process chamber 108 and workpiece holder 150 or for re-sterilization after a contamination event. When an ultraviolet radiation source is used, the interior of the process chamber 108 may contain considerable reflective metal to reflect the ultraviolet radiation throughout the interior of the process chamber 108.

A loading/unloading/packaging environment 316 is coupled to the process chamber 108 by an isolation valve 314. Isolation valve 314 has an open state and a closed state. When isolation valve 314 is open, workpieces to be sterilized may be moved from the loading/unloading/packaging environment 316 to the workpiece holder 150 for GCIB sterilization. Likewise, sterilized workpieces can be moved from the workpiece holder 150 to the loading/unloading/packaging environment 316 for sterile packaging before removal from the sterilizing system 300. Conventional mechanisms and/or robotic handlers may perform the transfers and packaging of the workpiece.

In typical operation, the process chamber 108 of the sterilizing system 300 is initially cleaned and then initially sterilized. Initial sterilization of the process chamber 108, and mechanisms therein including the workpiece holder 150 may be done by evacuating process chamber 108, then closing the valves 304, 312, 302, and 314 and introducing a sterilant gas 320 to the process chamber through valve 318. After allowing adequate time for sterilization, the valve 318 may be closed and the sterilant gas evacuated from the process chamber 108 by opening isolation valve 304 and evacuating the process chamber 108 using vacuum system 306. Alternatively, the interior of the process chamber 108 and mechanisms contained therein including the workpiece holder 150 may be initially sterilized by closing valves 312, 302, 318, and 314 and evacuating the process chamber 108 through isolation valve 304 using vacuum system 306—then by activating radiation source 322, which may be a short-wave ultraviolet radiation source, to sterilize the process chamber 108 and mechanisms therein.

After initial sterilization of the process chamber 108, one or more workpiece(s) 10 to be sterilized may be loaded sequentially or in parallel onto the workpiece holder 150, evacuated, and irradiated by GCIB or Neutral Beam (128, 1128, 1148, or 1314). The process chamber 108 may then be vented to atmospheric pressure using a sterile venting gas 308, and the workpiece 10 then unloaded to the loading/unloading/packaging environment 316 for packaging and/or removal from the sterilizing system 300. The loading/unloading/packaging environment 316 may enable direct insertion of sterilized work pieces into sterile containers. The load-sterilize-unload cycle may be repeated as many times as required for the sterilization job at hand.

The workpiece 10 is not exposed to sterilant gas 320 nor to radiation source 322, but rather is only sterilized by GCIB or Neutral Beam (128, 1128, 1148, or 1314), avoiding exposure to toxic materials and/or undesirable effects of radiation or other sterilizing methods. The sterilization that is performed via the present invention may also be limited to certain areas to further prevent any adverse affects on the finished product from this very process.

Gas-cluster ion-beam processing or Neutral Beam processing may be used to perform in-situ or post-process sterilization of medical devices with specific sterilization process needs. Certain situations where other known sterilization techniques such as UV light, high temperature exposure, or wet method processing are not suitable can benefit from use of this new alternative method. Surface-only processing makes this technology attractive when compared to other methods that may cause product damage or create unwanted degradation by damaging the subsurface regions that are not a source of bio-contamination. GCIB or Neutral Beam sterilization (as a final in-situ step), in combination with other beam surface processing step(s), in particular beam-induced or beam-assisted drug deposition application steps, etching steps, smoothing steps, etc., make this technology particularly useful and advantageous. In such applications, the initially sterilized process chamber 108 is loaded with the workpiece 10, multiple GCIB or Neutral Beam processing steps including a sterilizing step are preformed, and the finished product removed and optionally packaged. For sterilization of insulating or high electrical resistivity material surfaces, Neutral Beam processing may be preferred over GCIB.

Specific applications of the present invention include drug eluting implants and implants having areas adapted for enhanced cell growth. Drug eluting implants, such as stents, which finely control the area of coated drugs can be created using the present invention. Implants with areas adapted for enhanced cell growth using GCIB or Neutral Beam processing can be sterilized as part of the process to further reduce any risk of contamination.

The advantages of using GCIB or Neutral Beam processing are numerous and can be generalized as follows: First, the processing is carried out in a vacuum environment which provides complete environmental control over biological contamination and provides safe storage until the packaging process can begin. Second, the GCIB and Neutral Beam processing affects only a shallow surface layer, leaving the underlying material undamaged and creating no sub-surface damage or degradation. Third, GCIB or Neutral Beam allows sterilization of the immediate surface without significantly heating the bulk material, thus allowing sterilization of temperature-sensitive materials at approximately ordinary room temperatures. Another benefit of GCIB or Neutral Beam sterilization is the avoidance of ultraviolet, x-ray, or gamma ray, or other types of damage caused by other conventional techniques that can cause degradation of many materials. The combination or individual merits of these advantages may make GCIB or Neutral Beam sterilization attractive for situations that cannot tolerate wet processing, ultraviolet exposure or oxidative environments or situations where environmental control is difficult prior to packaging.

While GCIB and the use of Neutral Beams derived from GCIB has advantages in many applications, there are also limitations that must be considered before choosing such sterilization processing. First, the product for sterilization must be vacuum compatible. This means that the product must be able to withstand the rigors of the vacuum process without damage, and that the product is compatible with a vacuum level suitable for beam processing. Further, it is important that this vacuum level can be maintained while processing without excessive product out-gassing that may adversely affect the process. Lastly, beam processing is a “line of sight” process, which means that all surfaces of the sample that are intended to be sterilized must be exposed to the beam for the process to work. Depending on the shape and complexity of the object being sterilized, this may require very elaborate fixtures and manipulation tools and may prove to be impractical or impossible for some complex shapes. For many shapes and geometries, the required multiple exposures can be readily accomplished by manipulating, rotating, articulating, and/or repositioning the object during processing using conventional holding mechanisms that will be readily known by those skilled in the art.

### Exemplary Embodiment

Titanium was selected as an exemplary substrate for evaluation of GCIB sterilization since titanium is one of several commonly employed materials for implantable medical devices and prostheses. Since titanium is not an electrical insulator, GCIB is a practical choice for processing. Titanium foil was cut into pieces of approximately 1.5 cm×1.5 cm square. The cut pieces of titanium foil were openly exposed to ambient atmosphere in an inhabited area for 24 hours to promote the incidence of bacteria and/or bacterial spores to attach to the surface of the titanium foil squares. Following ambient exposure, Group 1 of the titanium foil squares was treated with argon GCIB irradiation at 30 kV acceleration potential with 5×10¹⁴ ions/cm² dose on both sides, for a total GCIB irradiation time of 90 seconds. Following ambient exposure, Group 2 was sterilized using a conventional sterilization process by being placed in a sterilization pouch and subjected to 20 minutes in a Harvey® Chemiclave 5000 sterilizer with Harvey® Vapo-Sterile solution. As a control, Group 3 was not further treated after the exposure to ambient atmosphere. Foil from each group was placed in individual pre-warmed LB-Agar (Luria Bertani Agar, a general purpose, non preferential, bacterial culture medium) plates (Sigma L5542) and placed in a 37° C. incubator for 72 hours and bacterial colonies were visually quantified.

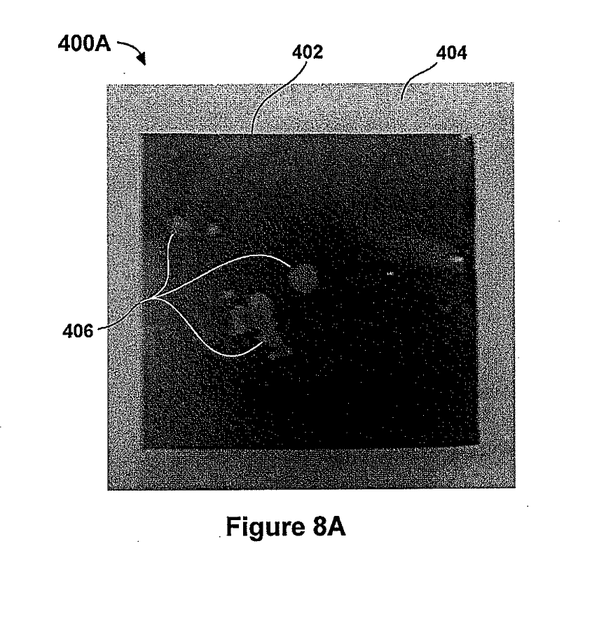

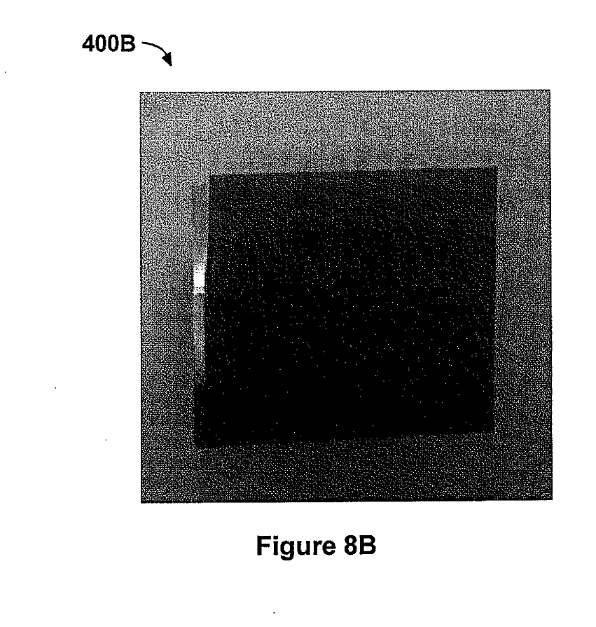

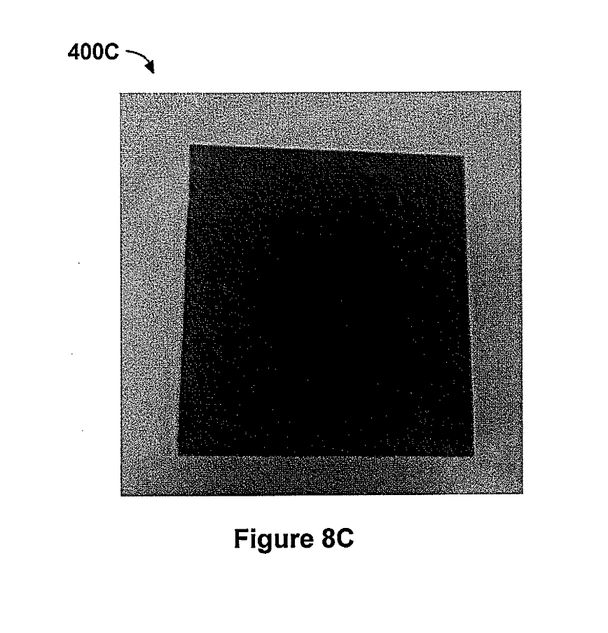

FIG. 8A shows a photograph 400A of a Group 3 (control group) titanium foil piece 402 in agar medium 404 showing the presence of numerous bacterial colonies growing on the foil several exemplary bacterial colonies 406 are indicated on the photograph.

FIG. 8B shows a photograph 400B of a Group 2 (conventionally sterilized) titanium foil piece in agar medium showing complete absence of bacterial colonies, indicating sterilization after ambient exposure.

FIG. 8C shows a photograph 400C of a Group 1 (GCIB sterilized) titanium foil piece in agar medium, again showing complete absence of bacterial colonies, indicating the effectiveness of the GCIB sterilization after ambient exposure.

Both Groups 1 and 2 had no bacterial colonies present, representing 0% surface area occupied by colonies. In comparison, the untreated control Group 3 had 27 visible bacterial colonies, several of which may have been the product of multiple colonies merging into a larger colony. All of the control Group 3 samples had visible bacterial colonies. None of the Group 1 or Group 2 samples had visible bacterial colonies. The total titanium surface covered by bacterial colonies for the control Group 3 was about 15%.

When the object to be sterilized is not an electrical conductor or is otherwise negatively sensitive to GCIB processing, then the use of an accelerated Neutral Beam derived from a GCIB is a preferred processing technique.

Although the invention has been described for exemplary purposes as using a Neutral Beam derived from a gas cluster ion beam for processing charge sensitive insulating materials, it is understood by the inventors that benefits obtained by application of such Neutral Beam surface processing is not limited to the specific materials discussed and that it offers improvements for many charge sensitive materials and electrically insulating or high resistivity materials, including without limitation, glass, quartz, sapphire, and polymers, including without limitation polystyrene, PTFE, PEEK, and PETE. It is understood that objects for medical implant benefit from Neutral Beam processing when formed from plastic or polymer or co-polymer materials including polyethylene and other inert plastics, solid resin materials, glassy materials, biological materials such as bone, collagen, silk and other natural fibers, various ceramics including titania, as well as other materials that may be suitable for the application and that are appropriately biocompatible and which are sensitive to charging or charge damage by ion beams.

Although the invention has been described with respect to various embodiments, it should be realized this invention is also capable of a wide variety of further and other embodiments within the spirit and scope of the invention and the appended claims.

It is claimed:

1. A method for sterilizing a workpiece, comprising the steps of:
providing a reduced pressure chamber;
forming gas-cluster ion-beam comprising gas cluster ions in the reduced pressure chamber;
accelerating the gas cluster ions;
providing conditions that permit or cause at least partial fragmentation or dissociation of at least part of the accelerated gas cluster ions in the gas cluster ion beam;
removing charged particles from the gas cluster ion beam containing at least partially fragmented or dissociated gas cluster ion beam to form an accelerated Neutral Beam having a path in the reduced pressure chamber;
providing a workpiece holder in the reduced pressure chamber for holding the workpiece in the Neutral Beam path;
irradiating at least a portion of a surface of the workpiece with the accelerated Neutral Beam for sterilizing the portion.

2. The method of claim 1, wherein the forming step includes accelerating the gas-cluster ion-beam using an acceleration potential of at least 2 kV.

3. The method of claim 1, wherein the forming step comprises forming a gas-cluster ion-beam comprising a noble gas or a mixture of a noble gas with oxygen.

4. The method of claim 1, wherein the at least a portion of a surface is an entire surface.

5. The method of claim 1, wherein the providing a workpiece holder step further comprises sterilizing the workpiece holder.

6. A method for sterilizing a workpiece, comprising the steps of:
a. providing a chamber having an interior and a workpiece holder in the interior;
b. initially sterilizing the workpiece holder and the interior of the chamber;
c. loading a workpiece onto the workpiece holder to be held thereby for sterilization;
d. reducing the pressure in the chamber;
e. forming an accelerated gas-cluster ion-beam;
f. providing conditions that permit or cause at least partial fragmentation or dissociation of the accelerated gas cluster ion beam;
g. removing charged particles from the at least partially fragmented or dissociated accelerated gas cluster ion beam to form an accelerated Neutral Beam;
h. directing the accelerated Neutral Beam onto the workpiece;
i. irradiating at least a portion of a surface of the workpiece with the accelerated neutral beam;
j. discontinuing irradiation when the at least a portion of a surface of the workpiece has received a predetermined dose; and
k. unloading the workpiece from the workpiece holder and removing it from the chamber.

7. The method of claim 6, further comprising the step of:
venting the chamber with a sterile gas.

8. The method of claim 6, wherein the at least a portion of a surface is an entire surface.

9. The method of claim 6, wherein the step of unloading includes placing the workpiece directly into a sterile container.
